(19) United States
(12) Patent Application Publication
(10) Pub. No.: US 2025/0090278 A1
(43) Pub. Date: Mar. 20, 2025

(54) MAGNESIUM MESH WITH FEATURES DESIGNED TO BIOABSORB PROGRESSIVELY TO IMPROVE DENTAL BONE REGENERATION

(71) Applicant: UNIVERSITY OF PITTSBURGH - OF THE COMMONWEALTH SYSTEM OF HIGHER EDUCATION, Pittsburgh, PA (US)

(72) Inventors: Andrew J. BROWN, Pittsburgh, PA (US); Charles S. SFEIR, Coraopolis, PA (US); Kelly Bolden WILLIAMS, Coraopolis, PA (US)

(21) Appl. No.: 18/580,805

(22) PCT Filed: Jul. 18, 2022
(86) PCT No.: PCT/US2022/037461

§ 371 Date: Jan. 19, 2024

Related U.S. Application Data
(60) Provisional application No. 63/223,774, filed on Jul. 20, 2021.

(51) Int. Cl. A61C 8/02; A61L 27/04; A61L 27/58

(52) U.S. Cl. CPC A61C 8/0006; A61L 27/04; A61L 27/58; A61L 2430/12

(57) ABSTRACT

The invention relates to dental bone grafting devices and magnesium meshes having features that are designed to corrode and/or absorb progressively, e.g., in stages, in order to improve dental bone regeneration, as well as methods for preparing the meshes. The meshes include a framework, and a geometric design is formed within the framework that includes design features. The geometric design and design features are selected and manipulated to provide the progressive corrosion and/or absorption profile of the mesh.

### CROSS-REFERENCE TO RELATED APPLICATIONS

This patent application claims the benefit of U.S. Provisional Patent Application Ser. No. 63/223,774 filed on Jul. 20, 2021, and entitled “A Magnesium Mesh with Features Designed to Resorb Progressively to Improve Dental Bone Regeneration”, the contents of which are incorporated herein by reference.

### GOVERNMENT SUPPORT STATEMENT

The invention was made with government support under DE026915 awarded by the National Institutes of Health (NIH). The government has certain rights in the invention.

### FIELD OF THE INVENTION

The invention includes dental bone grafting devices and magnesium meshes having features that are designed to absorb progressively in order to improve dental bone regeneration, and methods for preparing the meshes. The meshes are particularly useful in dental bone grafting procedures.

### BACKGROUND

Over one million dental bone grafting procedures are performed annually in the United States, most frequently in advance of dental implant placement to restore the function and aesthetics of missing teeth. Dental bone grafting procedures are generally associated with positive outcomes. However, in the thirty percent of cases where there has been a significant amount of bone loss or where patients have comorbidities that make bone regeneration more challenging, the end result can be unpredictable and unreliable resulting in success rates as low as sixty percent requiring revision procedures.

Maximizing dental bone regeneration in these major bone grafting procedures is currently attempted using form-stable barrier membranes or meshes, that can protect the healing site from mechanical insults for up to nine months. Unfortunately, existing form-stable barrier membranes and meshes require an invasive removal procedure prior to dental implant placement and exhibit complication rates of up to forty-four percent, which decreases the likelihood of achieving optimal grafting outcomes.

In general, magnesium-based implants have been explored for many medical applications due to their combination of mechanical strength and bioabsorbable or absorbable properties. Magnesium-based devices have been used clinically for fracture fixation and stabilization, sports medicine applications, and cardiovascular applications. Known magnesium meshes, with geometric designs based on other resorbable or absorbable materials or based on the design of non-resorbable materials (e.g., titanium meshes), are likely to fail in areas of high stress in situ as the magnesium absorbs. These high stress areas include points where the mesh is fixated by a screw, areas where the mesh is bent to adapt to the surrounding bone, and high energy areas containing sharp feature intersections. Additionally, the in-situ environment has complex fluid flows and mechanical insults that result in non-ideal absorption of the magnesium material unlike what is observed in in-vitro or in-silico experiments. These factors combine to threaten the stability of a magnesium mesh used in a guided bone regeneration application, thus jeopardizing bone healing.

Thus, oral surgeons and periodontists lack a form-stable, yet absorbable membrane or mesh that can maximize bone regeneration, reduce the morbidity and time associated with device removal, and ultimately reduce the likelihood that expensive and time-consuming revision procedures are needed. Magnesium's in situ absorption mechanism of surface corrosion requires unique design features not incorporated into non-resorbable meshes or polymers with bulk absorption properties to achieve these clinical properties.

There is a need in the art to develop a magnesium-based mesh that provides the form-stability necessary for optimal bone healing with the absorbability necessary to eliminate the device removal procedure and improve soft tissue response.

### SUMMARY OF THE INVENTION

In one aspect, the invention provides a dental bone grafting device that includes a mesh, that includes a framework fabricated of a material selected from the group consisting of magnesium metal and a magnesium-based metal alloy; and a geometric design formed within the framework comprising a plurality of design features, that include two or more fixation point openings each having a corresponding fixation connecting feature corresponding thereto; and one or more non-fixation point openings each having a corresponding non-fixation connecting feature corresponding thereto, wherein each of the corresponding fixation connecting feature and the corresponding non-fixation connecting feature surrounds and/or connects each of the two or more fixation point openings and the one or more non-fixation point openings, respectively, and wherein each of the corresponding fixation connecting feature and the corresponding non-fixation connecting feature has a size that is structured to provide progressive and/or staged corrosion and/or absorption of the mesh.

In certain embodiments, each of the two or more fixation point openings and the one or more non-fixation point openings, and the corresponding fixation connecting feature and the corresponding non-fixation connecting feature corresponding thereto, respectively, has a corresponding size. The size of each of the corresponding fixation connecting feature and the corresponding non-fixation connecting feature can be selected from the group consisting of surface area, width, and thickness. The surface area of the corresponding fixation connecting feature of the two or more fixation point openings can be greater than the surface area of the corresponding non-fixation connecting feature of the one or more non-fixation point openings. The corresponding fixation connecting feature that surrounds the two or more fixation point openings can absorb or corrode over a longer period of time as compared to the corresponding non-fixation connecting feature that surrounds the one or more non-fixation point openings.

In certain embodiments, the two or more fixation point openings are structured to receive a fastening device. The fastening device can be selected from the group consisting of a bolt, screw, tack, and pin.

In certain embodiments, the magnesium-based alloy includes from about 0.85 to about 1.4 weight percent zinc; from about 0.2 to about 0.5 weight percent calcium; from about 0.2 to about 0.5% weight percent manganese; and a balance of magnesium based on a total weight percent of the magnesium-based alloy.

In certain embodiments, the framework is a magnesium foil. The magnesium foil can have a thickness from about 0.15 to about 0.55, or about 0.35 mm.

In certain embodiments, the two or more fixation point openings are positioned along the perimeter of the framework. The size of the corresponding fixation connecting feature of the two or more fixation point openings can be greater at the interface of the fastening device and the magnesium framework as compared to another portion of the two or more fixation point openings. There can be at least two different sizes of the corresponding fixation connecting feature and/or corresponding non-fixation connecting mesh to promote a progressive and/or staged corrosion and/or absorption profile.

In another aspect, the invention includes a method of making a dental bone grafting device. The method includes fabricating a mesh, which includes preparing a framework composed of a material selected from the group consisting of magnesium metal and a magnesium-based metal alloy; and forming a geometric design within the framework comprising a plurality of design features, including two or more fixation point openings each having a corresponding fixation connecting feature corresponding thereto; and one or more non-fixation point openings each having a corresponding non-fixation connecting feature corresponding thereto, wherein each of the corresponding fixation connecting feature and the corresponding non-fixation connecting feature surrounds and/or connects each of the two or more fixation point openings and the one or more non-fixation point openings, respectively, and wherein each of the corresponding fixation connecting feature and the corresponding non-fixation connecting feature has a size that is structured to provide progressive and/or staged corrosion and/or absorption of the mesh.

In another aspect, the invention includes a mesh that includes a framework fabricated of a material selected from the group consisting of magnesium metal and a magnesium-based metal alloy; and a geometric design formed within the framework comprising a plurality of design features, including two or more fixation point openings each having a corresponding fixation connecting feature corresponding thereto; and one or more non-fixation point openings each having a corresponding non-fixation connecting feature corresponding thereto, wherein each of the corresponding fixation connecting feature and the corresponding non-fixation connecting feature surrounds and/or connects each of the two or more fixation point openings and the one or more non-fixation point openings, respectively, and wherein each of the corresponding fixation connecting feature and the corresponding non-fixation connecting feature has a size that is structured to provide progressive and/or staged corrosion and/or absorption of the mesh.

In yet another aspect, the invention includes a method of making a mesh. The method includes preparing a framework composed of a material selected from the group consisting of magnesium metal and a magnesium-based metal alloy; and forming a geometric design within the framework comprising a plurality of design features, including two or more fixation point openings each having a corresponding fixation connecting feature corresponding thereto; and one or more non-fixation point openings each having a corresponding non-fixation connecting feature corresponding thereto, wherein each of the corresponding fixation connecting feature and the corresponding non-fixation connecting feature surrounds and/or connects each of the two or more fixation point openings and the one or more non-fixation point openings, respectively, and wherein each of the corresponding fixation connecting feature and the corresponding non-fixation connecting feature has a size that is structured to provide progressive and/or staged corrosion and/or resorption of the mesh.

### BRIEF DESCRIPTION OF THE DRAWINGS

A full understanding of the invention can be gained from the following description of the preferred embodiments when read in conjunction with the accompanying drawings, as follows:

FIG. 1 is an image that illustrates a base framework design of a magnesium mesh, in accordance with certain embodiments of the invention;

FIG. 2 is an image that illustrates a larger framework design of a magnesium mesh as compared to FIG. 1, in accordance with certain embodiments of the invention;

FIGS. 3A and 3B are images that each illustrates a faster corroding and/or absorbing framework design of a magnesium mesh, achieved by reducing the surface area of perimeter connecting features and interior connecting features, as compared to FIGS. 1 and 2, in accordance with certain embodiments of the invention;

FIG. 4 is an image that illustrates a tapered design of a magnesium mesh as compared to FIGS. 1, 2, 3A and 3B, in accordance with certain embodiments of the invention;

FIG. 5 is a photograph that illustrates a set-up used in an experiment to measure in vitro corrosion and resulting partially corroded magnesium meshes, in accordance with certain embodiments of the invention;

FIGS. 6A and 6B are images that each illustrates in vivo corrosion and/or absorption of a framework design of a magnesium mesh, in accordance with certain embodiments of the invention;

FIG. 7 is a plot that illustrates the results of a microCT assessment of partially corroded and/or absorbed screws and magnesium foil mesh frameworks, in accordance with certain embodiments of the invention;

FIGS. 8A and 8B are images that illustrate the results of a microCT and histological assessment completed on eight explanted samples of magnesium foil mesh frames with fixation screws, in accordance with certain embodiments of the invention; and

FIG. 9A is a plot that illustrates the weight of four different designs of the magnesium foil mesh devices remaining following in-vitro corrosion for 0 days, 4 days and 11 days, and FIG. 9B includes images illustrating that structural stability of the magnesium mesh designs was maintained through 11 days in-vitro, in accordance with certain embodiments of the invention.

### DETAILED DESCRIPTION OF THE PREFERRED EMBODIMENTS

The invention relates to dental bone grafting devices and meshes constructed or composed of a magnesium material that exhibit features, which are designed to corrode and/or absorb progressively or in stages, in order to improve dental guided bone regeneration. The magnesium material is selected from magnesium metal and magnesium metal alloys (e.g., magnesium-based alloys) or any combination thereof. In certain embodiments, the magnesium material is a magnesium metal alloy including magnesium as the base metal in combination with other metals (alloying elements), such as, but not limited to, zinc, calcium and manganese or any combination thereof. In certain embodiments, the magnesium metal alloy includes from about 0.85 to about 1.4 weight percent zinc, from about 0.2 to about 0.5 weight percent calcium, from about 0.2 to about 0.5 weight percent manganese, and a balance of magnesium based on the total weight of the alloy.

In certain embodiments, the magnesium material is in the form of a magnesium foil. The thickness of the magnesium foil varies and, in certain embodiments, is from about 0.15 to about 0.55 mm, or about 0.35 mm. In certain embodiments, the thickness of the magnesium material, e.g., magnesium foil, is uniform. In certain other embodiments, the thickness of the magnesium material, e.g., magnesium foil, is non-uniform. In general, the greater the thickness of the magnesium material, e.g., foil, the longer the amount of time for corrosion and absorption to occur when, for example, placed and fixed in a human body, e.g., for dental bone regeneration applications. A thick or thin magnesium foil is selected for an overall long or short absorption time, respectively; in addition, geometric design features are applied or incorporated to provide a progressive or staged absorption profile and maintain structural integrity and stabilization for a long and/or short period throughout the absorption process.

The progressive or staged corrosion and/or absorption of the magnesium material is controlled or tuned by the geometric design and/or features of the mesh. For instance, by changing, specifying and/or modulating the geometric design and/or features, the corrosion and/or absorption profile is controlled or tuned. Corrosion and/or absorption of the mesh is not uniform. For those parts or portions of the magnesium mesh where slow absorption is desired or beneficial, the geometric design and/or features of the specific mesh part or portion is designed to result in slower absorption as compared to other specific parts or portions. Specifically, the surface area (i.e., width) of a feature is increased when slower corrosion (and slower absorption) is desired as compared to other features, and decreased when faster corrosion (and faster absorption) is desired as compared to other features.

The meshes according to the invention are placed and fixed around a tooth and/or dental implant, or a completely edentulous area. The meshes include high stress areas, which include (i) points (e.g., fixation points) where the mesh is fixed to a substrate (such as surrounding bone) by a fastening device (e.g., one or more screws, tacks, pins, and the like), (ii) areas where the mesh is bent to adapt to the surrounding bone, and (iii) high energy areas containing sharp feature intersections. Additionally, the in-situ environment has complex fluid flows and mechanical insults that result in non-ideal absorption of the magnesium material, unlike what is seen in-vitro or in-silico. These factors combine to threaten the stability of a magnesium mesh used in a bone regeneration application, thus jeopardizing bone healing. In applications where resorbable or absorbable implants may be used (e.g., fracture fixation), the total resorption or absorption time of the device may be extended to provide mechanical stability for a longer (e.g., long) time period. In dental bone grafting procedures, the area where meshes are implanted are most frequently surgically re-entered six to nine months later for the placement of a dental implant. Thus, devices intended for use as resorbable or absorbable dental regenerative materials (e.g., meshes and screws) must balance maintaining structural integrity for long enough to support bone regeneration while not adversely impacting the second surgical re-entry for dental implant placement. The mesh design in accordance with the invention ensures that the structural stability and fixation of the mesh is maintained throughout bone healing, and allows points for fixation of the mesh to surrounding bone. In addition, the mesh design permits adaption to complex three-dimensional geometries observed in alveolar ridge regeneration.

The progressive corrosion and/or absorption of the magnesium material provides the ability for certain parts, portions, and/or features of the mesh to absorb faster or slower, e.g., in a shorter or longer period of time, respectively, than certain other parts, portions, and/or features of the mesh. In some embodiments, it is beneficial for absorption of the periphery or perimeter or circumference of the mesh to occur over a longer time period as compared to the center portion and/or features of the mesh (which is surrounded or encompassed by the periphery or perimeter or circumference). It is typical for multiple, e.g., two or more, fixation points to be located/positioned along the periphery or perimeter or circumference of the mesh. When the periphery or perimeter or circumference of the mesh is intact for a longer time period, it provides a stable-form to maintain the structural integrity of the mesh and prevents early structural failure of the mesh, such as, in dental bone regeneration applications.

The meshes according to the invention include a framework (substrate or matrix) fabricated of the magnesium material (magnesium metal or a magnesium-based alloy), such as, a magnesium foil. A geometric design and/or features are formed within the framework. The methods and apparatus employed to form the geometric design and/or features include those known in the art for such purpose. In certain embodiments, the mesh is produced using laser cutting, extrusion, sintering, or expanded metal manufacturing. The geometric design and/or features include multiple, e.g., two or more, fixation points, each having an opening (i.e., fixation point opening) formed in the framework, e.g., to receive a fastening device, and one or more non-fixation point openings formed in the framework to enable easy adaptation to complex three-dimensional bone defects. In certain embodiments, the geometric design and/or features include more than two fixation points. The openings are cut into, e.g., cut through, the framework (e.g., magnesium foil) such that a part, portion, or feature of the framework surrounds each of the fixation point openings and the non-fixation point openings, e.g., corresponding fixation connecting feature and corresponding non-fixation connecting feature, respectively, and connects each of the two or more fixation point openings and the one or more non-fixation point openings. In certain embodiments, the fixation point openings are formed along the periphery or perimeter of the framework, and a plurality of non-fixation point openings are formed within the interior portion of the framework that is surrounded by the fixation point openings. The corresponding fixation and/or corresponding non-fixation connecting features have varying surface areas, e.g., horizontal widths, surrounding the openings such that it is structured to provide the progressive/staged corrosion or absorption of the mesh.

The fixation points of the mesh are effective to maintain the mesh in a fixed location or position and therefore, the occurrence of slow corrosion or absorption is advantageous at these fixation points. The fixation points include, for example, bolts, screws, tacks, pins or other fastening devices that are used to connect the mesh to a surface, e.g., surrounding human bone or tissue. It is typically beneficial for the fixation points to corrode and absorb at a slower rate as compared to other parts, portions, or features, e.g., the interior parts, portions, or features, in order to prevent mechanical failure of the device. Thus, in certain embodiments, the features that extend around and/or between the fixation points/fixation point openings (e.g., the corresponding fixation connecting features) have a larger surface area (or horizontal width) as compared to the corresponding non-fixation connecting features that surround and/or connect non-fixation point openings in the mesh. For a larger surface area (or horizontal width), when forming the openings, they are spaced a farther distance apart; whereas for a smaller surface area (or horizontal width), when forming the openings, they are spaced a closer distance apart.

Each of the two or more fixation point openings and the one or more non-fixation point openings, and the corresponding fixation connecting features and the corresponding non-fixation connecting features corresponding thereto, has a corresponding size. The size of the corresponding fixation connecting features or the corresponding non-fixation connecting features consists of one or more of its surface area, width (horizontal thickness), and height (vertical thickness). In certain embodiments, the width (horizontal thickness) of the corresponding fixation connecting features of the two or more fixation points/fixation point openings is greater than the width (horizontal thickness) of the corresponding non-fixation connecting features of non-fixation point openings. The corresponding fixation connecting features that surround the two or more fixation points/fixation point openings corrodes and/or absorbs over a longer period of time as compared to the corresponding non-fixation connecting features that surround the non-fixation point openings. The two or more fixation point openings are structured to receive a fastening device that includes those that are known in the art for such a purpose, including but not limited to a bolt or screw.

In certain embodiments, the two or more fixation points/fixation point openings are positioned along the perimeter of the mesh. The size of the corresponding fixation connecting features that surrounds and/or connects each of the two or more fixation points/fixation point openings is greater at the interface of the bolt/screw and the framework, as compared to another portion of the two or more fixation points.

In certain embodiments, there are at least two different sizes of the corresponding fixation connecting features and/or corresponding non-fixation connecting features that surround the fixation point openings and non-fixation point openings present in the framework, in order to promote a progressive/staged corrosion or absorption profile. For instance, the corresponding fixation connecting features that surround the fixation point openings are greater than the corresponding non-fixation connecting features that surround the non-fixation point openings.

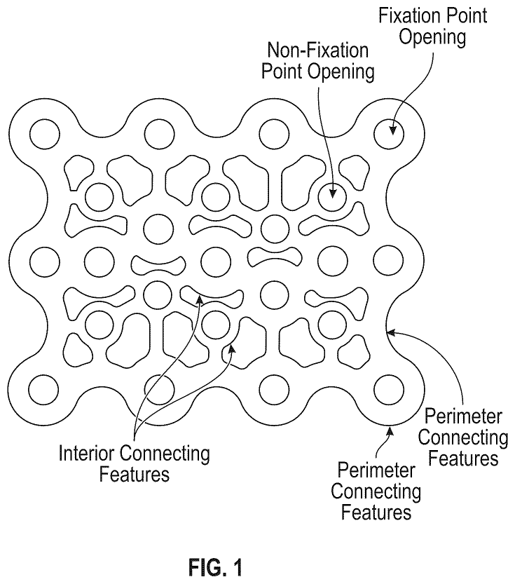

FIG. 1 is an image that shows a magnesium foil mesh in accordance with certain embodiments of the invention. The mesh includes a framework or substrate constructed or composed of the magnesium foil material having openings or apertures (geometric features) formed therein. In certain embodiments, the geometric features of the framework provide a repeating or non-repeating pattern. The mesh shown in FIG. 1 is in a rectangular shape and has smooth, curved edges along its perimeter. As shown in FIG. 1, the periphery or perimeter includes ten fixation point openings (light shaded circular holes) connected together by the fixation connecting features (dark shaded areas) surrounding the openings, e.g., perimeter connecting features. The mesh also includes a plurality of non-fixation point openings (e.g., light shaded circular holes formed within the interior portion of the mesh) connected together by the non-fixation connecting features (dark shaded areas) surrounding the openings, e.g., interior connecting features. In FIG. 1, the non-fixation point openings in the mesh are surrounded by the fixation point openings. The openings formed in the framework vary in size and shape. In addition, the framework that extends around and/or between the openings, e.g., the connecting features (dark shaded area shown in FIG. 1), such as to surround the openings and connect them, also varies in size. As used herein, the size (of the features that extend between the openings, e.g., the connecting features) refers to the surface area or the width along the horizontal surface of the framework. In FIG. 1, the corresponding fixation connecting features that extend around and between the ten fixation point openings (that are positioned on the perimeter of the framework) have a larger size than the corresponding non-fixation connecting features that extend around and between some of the circular openings that are formed in the interior portion of the framework. The corresponding fixation connecting features and/or corresponding non-fixation connecting features (e.g., the dark shaded portion shown in FIG. 1) have varying surface areas, e.g., horizontal widths, surrounding the openings such that it is structured to provide the progressive/staged corrosion or absorption of the mesh. According to the invention, the parts, portions, or features of the framework that have a larger surface area (horizontal width) corrode slower (i.e., take more time) as compared to other parts, portions, or features of the framework that have a smaller surface area (horizontal) and corrode and/or absorb faster (i.e., take less time). Additionally, where the intersection of features in the interior would produce a large surface area of magnesium, new voids or openings are created to maintain the desired resorption rate.

As shown in FIG. 1, the ten fixation point openings positioned along the perimeter of the mesh have corresponding fixation connecting features (e.g., perimeter connecting features) that are of a large size (as compared to the corresponding non-fixation connecting features (e.g., interior connecting features) of non-fixation point openings formed in the interior of the framework), that would corrode and absorb more slowly in order to provide structural stability and integrity to the mesh, e.g., when used as a dental bone grafting device. Further, in certain embodiments, the corresponding fixation connecting features at the fixation bolt/screw-magnesium framework interface is larger in size (e.g., surface area or width) as compared to other portions of the fixation point in order to overcome or mitigate the accelerated absorption that occurs from stress corrosion at the interface, including absorption that occurs in-situ.

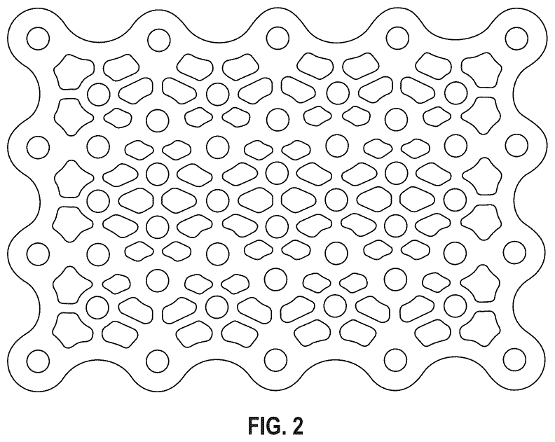

FIG. 2 is an image that shows a magnesium mesh having a larger framework design as compared to FIG. 1. In this design, there are two larger portions of the corresponding non-fixation connecting features to account for the larger size of the overall mesh and to mitigate the accelerated resorption that may occur.

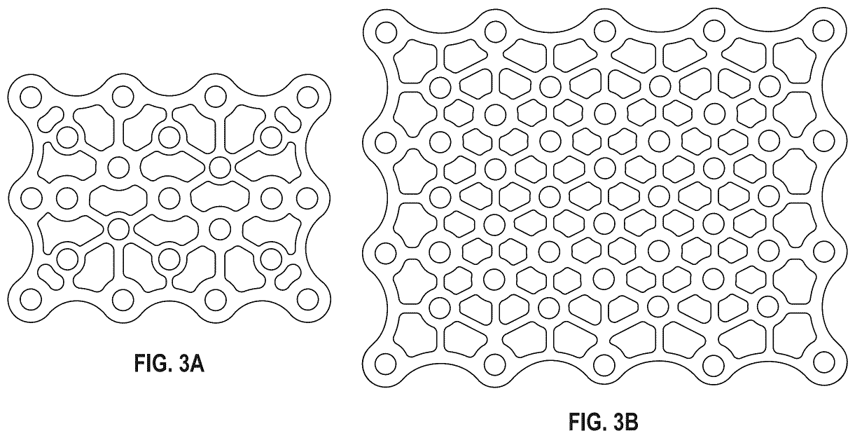

FIGS. 3A and 3B are images that each shows a magnesium mesh having a framework design that provides faster corrosion and absorption as compared to the framework designs in FIGS. 1 and 2. In FIG. 3A, smaller dimensions of the corresponding non-fixation connecting features are used relative to FIG. 1. When placed in situ, the mesh of FIG. 3A loses structural integrity and ultimately absorbs faster than the mesh in FIG. 1, which may be desired in certain clinical indications. Similarly, the mesh in FIG. 3B has smaller dimensions for the corresponding non-fixation connecting features relative to the mesh shown in FIG. 2.

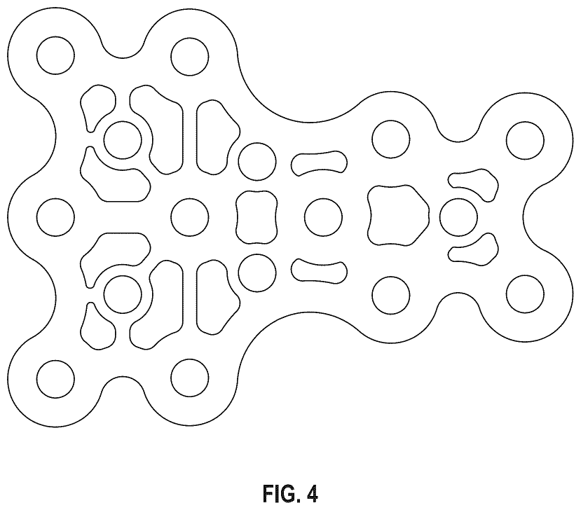

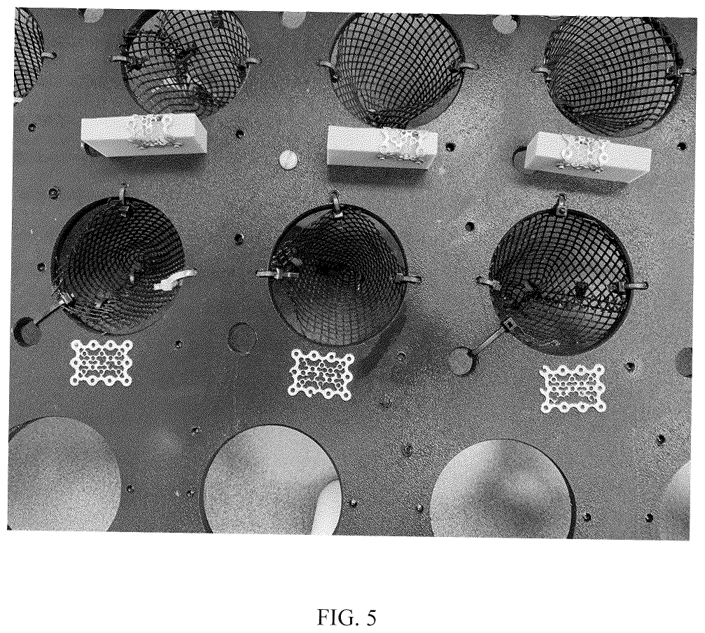

FIG. 4 is an image that shows a tapered framework design as compared to the rectangular framework designs in FIGS. 1, 2, 3A and 3B. In situations where trimming of the mesh with scissors or other devices may not be preferred, different geometries of the mesh may be produced to fit different bone defects within the jaw.

The magnesium meshes include redundant features (e.g., multiple corresponding fixation connecting features attaching fixation points, and multiple corresponding non-fixation connecting features attaching non-fixation point), such that the loss of mechanical integrity of a single feature (as occurs naturally during resorption) does not solely contribute to loss of stability of the entire membrane.

In certain embodiments, the fixation points are the features of the mesh framework that are designed to corrode and/or absorb the slowest. Accordingly, the corresponding fixation connecting features corresponding to the fixation points/fixation point openings have a large size, e.g., surface area and/or width.

The following mesh features corrode and/or absorb slowest to fastest (from a. to e., respectively):
a. Fixation points;
b. Perimeter connecting features connecting the fixation points/fixation point openings along the perimeter of the framework;
c. Interior connecting features closest to the occlusal plane (middle of membrane height) as these are subjected to the most prominent mechanical insults;
d. Interior connecting features on the height axis of the framework (as shown in FIG. 1) as this is the primary loading direction; and
e. Interior connecting features on the length axis of the framework.

In accordance with the invention, the geometric designs reduce sharp edges which harm soft tissue and serves as a nitus for early corrosion.

In certain embodiments, there are at least two distributions of corresponding fixation connecting features and/or corresponding non-fixation connecting feature sizes to promote a progressive/staged resorption profile with concomitant loss of mechanical integrity: (i) smaller or minor sized corresponding non-fixation connecting features that lose mechanical integrity in 4-6 weeks following placement; and (ii) larger or major corresponding fixation connecting features that lose mechanical integrity in 12-16 weeks following placement.

The above design concepts can be applied to a variety of overall mesh framework shapes and sizes. For example, mesh frameworks ranging from 10 mm×20 mm, to 40 mm×50 mm are currently clinically used. The designs according to the invention introduce new connections of the above features to account for changes in framework size while preventing early loss of structural integrity.

The above design concepts are applicable to different thicknesses and geometries of magnesium mesh for different clinical applications. For example, smaller bone regeneration procedures necessitate shorter periods for regeneration. In certain embodiments, the above design concepts are applied such that minor features lose mechanical integrity in 2-4 weeks and major features lose mechanical integrity in 8-12 weeks.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

### EXAMPLES

Mesh designs in accordance with certain embodiments of the invention were prepared using an alloy including 0.85 to 1.4 weight percent zinc, 0.2 to 0.5 weight percent calcium, 0.2 to 0.5 weight percent manganese, and a balance of magnesium based on the total weight of the alloy. The alloy was in the form of a magnesium-based foil, which had a uniform thickness (height) of 0.35 mm.

FIG. 5 shows a set-up for conducting the experiments, and the resulting meshes produced. The resulting meshes demonstrate that the middle portion of the mesh framework having a thinner (horizontally narrower) interior connecting mesh had increased corrosion as compared to corrosion of the perimeter of the mesh framework, which had thicker (horizontally wider) perimeter connecting mesh.

Meshes constructed with a geometry similar to that of FIG. 1 were manufactured using a magnesium alloy containing zinc, calcium, and manganese, and laser cutting to produce the desired geometric design with a uniform thickness of 0.35 mm. These mesh samples were either mounted to synthetic bone blocks (stressed) using magnesium screws placed through fixation point openings or left in a flat state (unstressed). All samples were then immersed in a synthetic body fluid for up to 16 days with routine monitoring. FIG. 5 shows stressed and unstressed samples after 5 days of immersion in simulated body fluid. Notably, corrosion of the material occurred in a non-uniform, non-ideal way (i.e., it would be expected for uniform surface corrosion to occur). Full thickness corrosion was observed in many of the interior features, as well as around some of the fixation points for both stressed and unstressed samples. It was found that for the stressed samples, more features (interior and perimeter connecting mesh) were lost in areas of high mechanical strength. These data suggest that without the redundant design and increased thickness of fixation points and some interior features, the stressed meshes would have lost structural integrity and no longer been fixated to the blocks.

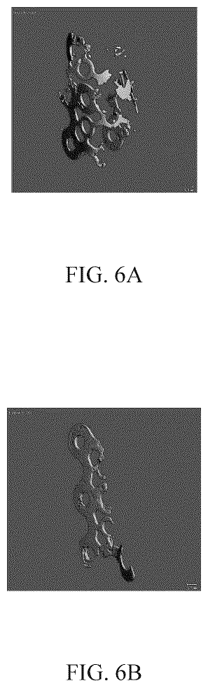

FIGS. 6A and 6B show partially corroded and/or absorbed mesh frameworks following 12 weeks of implantation in a canine experimental model in accordance with certain embodiments of the invention similar to the mesh design of FIG. 1.

Data generated in an in-vivo test bed validated that the thinner (horizontally narrower) corresponding non-fixation connecting features absorbed faster than the thicker (horizontally wider) corresponding fixation connecting features, enabling the structural integrity and fixation of the mesh to bone blocks to be sustained. Additionally, data generated in a canine ridge augmentation model also validated this finding. Fixation of the mesh to the bone was maintained throughout twelve weeks of healing, and there was no loss of structural integrity throughout the early healing phase. FIGS. 6A and 6B show that absorption occurs in a non-uniform, non-ideal manner in vivo, with more absorption occurring in areas of higher stress, but with maintenance of the thicker corresponding fixation features maintained through 12 weeks.

### MicroCT and Histological Assessment

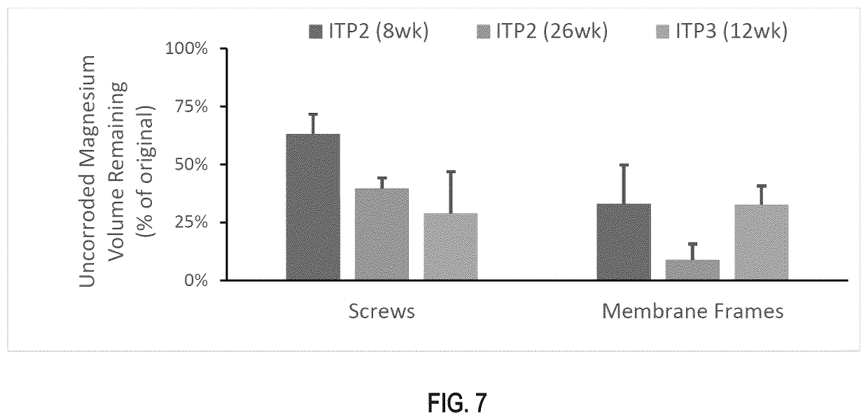

FIG. 7 illustrates the results of a microCT and histological assessment showing that magnesium screws absorbed over time and retained membrane fixation and stability, while magnesium foil mesh frames fabricated according to certain embodiments, similar to the design in FIG. 1, of the invention absorbed such that greater than 80% absorption occurred over a period of six months.

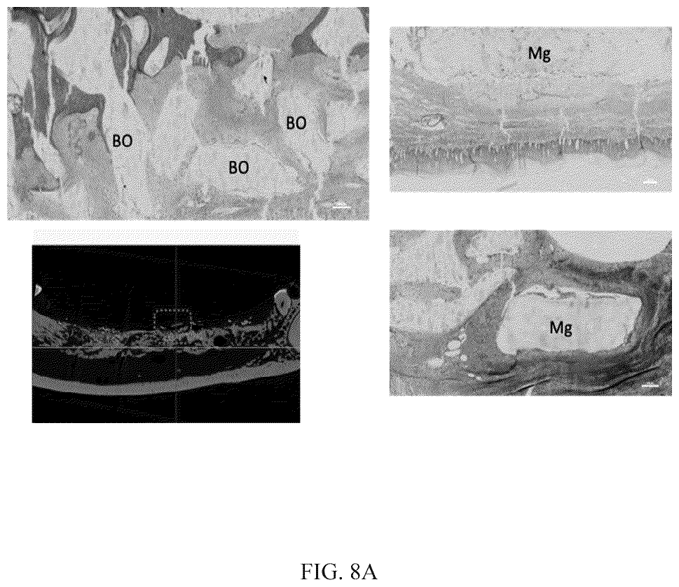

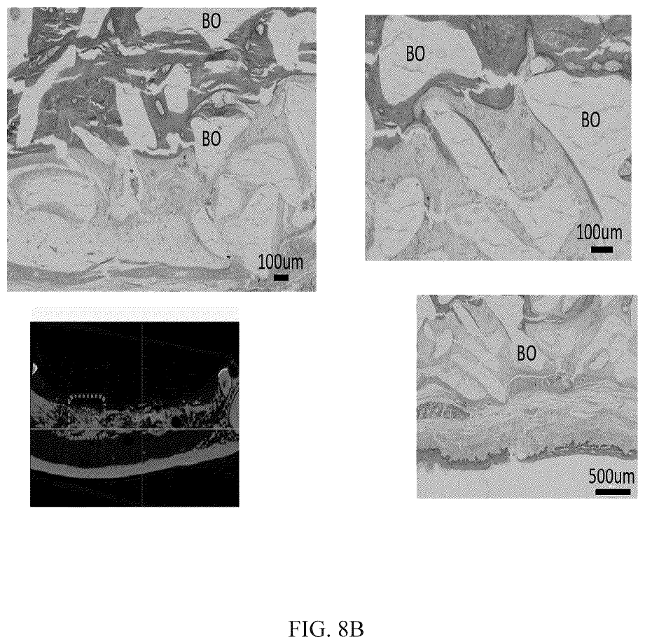

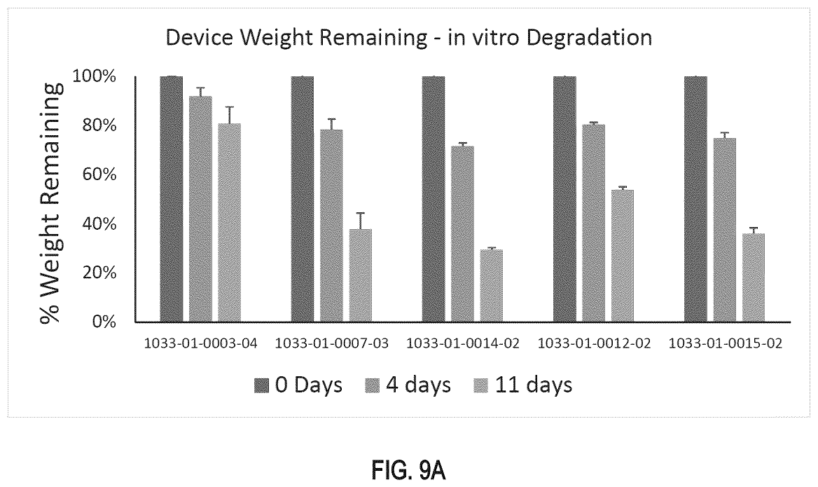

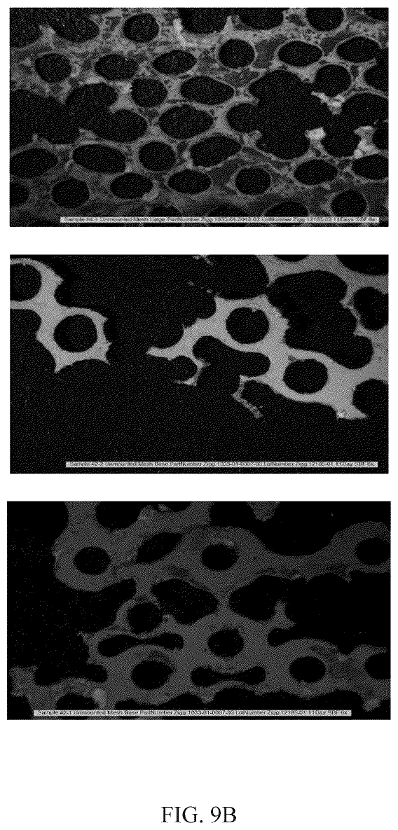

FIGS. 8A and 8B illustrate the results of a microCT and histological assessment completed on eight explanted samples of magnesium foil mesh frames with fixation screws fabricated in accordance with certain embodiments of the invention, similar to the design of FIG. 1, as compared to a control. For the inventive magnesium foil mesh frames, (i) bio-oss particles (designated as “BO” in FIG. 9A) were found to be integrated into bone with active osteoid, (ii) gingival tissue surrounded the magnesium frame components, and (iii) transitional area was identified around the magnesium fixation screws as bone regeneration occurred in the space previously occupied by the screw.

### Corrosion Testing

Corrosion testing was conducted for magnesium foil mesh frameworks with screws (corresponding to chart label 1033-01-0003-04) in accordance with certain embodiments of the invention, including those designs in FIG. 1 (corresponding to chart label 1033-01-0007-03), FIG. 2 (corresponding to chart label 1033-01-0012-02), FIG. 3a (corresponding to chart label 1033-01-0014-02), and FIG. 3b (corresponding to chart label 1033-01-0015-02). Generally, corrosion of the screws was significantly lower than all mesh corrosion, and smaller features (connecting mesh having smaller surface area) of the mesh were first to corrode. The distribution of different feature sizes prevented early loss of stability of the mesh; this is a result of the differences in surface area. Meshes of similar overall sizes and designs, but with smaller connecting feature surface areas exhibited faster overall corrosion (e.g., 1033-01-0014-02 vs. 1033-01-007-03 and 1033-01-0015-02 vs. 1033-01-0012-02). When mesh corrosion was normalized to surface area, corrosion rates of the meshes tested were within +/−10%.

FIG. 9A is a plot that illustrates the weight of the mesh device remaining following in-vitro corrosion for 0 days, 4 days and 11 days for various magnesium mesh designs in accordance with certain embodiments of the invention. FIG. 9B includes images illustrating that structural stability of the magnesium mesh designs was maintained through 11 days in-vitro even though there was less than 50% weight loss across all samples. The 11 day in-vitro time period was approximately equivalent to 12 weeks in-vivo.

Although data was generated using specific design parameters, the geometric design frameworks and meshes disclosed herein are applicable to other thicknesses of magnesium foil and other alloying systems. For example, a thicker magnesium foil may be selected for an overall longer corrosion or absorption time. The geometric design features are applied to provide the staged corrosion or absorption profile and maintain structural integrity and stabilization for a longer period throughout the absorption process.

We claim:

1. A dental bone grafting device, comprising:
a mesh, comprising:
a framework fabricated of a material selected from the group consisting of magnesium metal and a magnesium-based metal alloy; and
a geometric design formed within the framework comprising a plurality of design features, comprising:
two or more fixation point openings each having a corresponding fixation connecting feature corresponding thereto; and
one or more non-fixation point openings each having a corresponding non-fixation connecting feature corresponding thereto,
wherein each of the corresponding fixation connecting feature and the corresponding non-fixation connecting feature surrounds and/or connects each of the two or more fixation point openings and the one or more non-fixation point openings, respectively, and
wherein each of the corresponding fixation connecting feature and the corresponding non-fixation connecting feature has a size that is structured to provide progressive and/or staged corrosion and/or absorption of the mesh.

2. The dental bone grafting device of claim 1, wherein each of the two or more fixation point openings and the one or more non-fixation point openings, and the corresponding fixation connecting feature and the corresponding non-fixation connecting feature corresponding thereto, respectively, has a corresponding size.

3. The dental bone grafting device of claim 2, wherein the size of each of the corresponding fixation connecting feature and the corresponding non-fixation connecting feature is selected from the group consisting of surface area, width, and thickness.

4. The dental bone grafting device of claim 1, wherein the surface area of the corresponding fixation connecting feature of the two or more fixation point openings is greater than the surface area of the corresponding non-fixation connecting feature of the non-fixation point openings.

5. The dental bone grafting device of claim 4, wherein the corresponding fixation connecting mesh that surrounds the two or more fixation point openings corrodes or absorbs over a longer period of time as compared to the corresponding non-fixation connecting feature that surrounds the one or more non-fixation point openings.

6. The dental bone grafting device of claim 1, wherein the two or more fixation point openings are structured to receive a fastening device.

7. The dental bone grafting device of claim 6, wherein the fastening device is selected from the group consisting of a bolt and screw.

8. The dental bone grafting device of claim 1, wherein the magnesium-based metal alloy, comprises:
from about 0.85 to about 1.4 weight percent zinc;
from about 0.2 to about 0.5 weight percent calcium;
from about 0.2 to about 0.5% weight percent manganese; and
a balance of magnesium based on a total weight percent of the magnesium-based alloy.

9. The dental bone grafting device of claim 1, wherein the framework is a magnesium foil.

10. The dental bone grafting device of claim 9, wherein the magnesium foil has a thickness from about 0.15 mm to about 0.55 mm

11. The dental bone grafting device of claim 10, wherein the magnesium foil has a thickness of about 0.35 mm.

12. The dental bone grafting device of claim 1, wherein the two or more fixation point openings are positioned along the perimeter of the framework.

13. The dental bone grafting device of claim 7, wherein the size of the corresponding fixation connecting feature of the two or more fixation point openings is greater at the interface of the fastening device and the magnesium framework as compared to another portion of the two or more fixation point openings.

14. The dental bone grafting device of claim 1, wherein there are at least two different sizes of the corresponding fixation connecting feature and/or the corresponding non-fixation connecting feature to promote a progressive and/or staged corrosion and/or absorption profile.

15. The dental bone grafting device of claim 1, wherein the device comprises two or more fixation point openings.

16. A method of making a dental bone grafting device, comprising:
fabricating a mesh, comprising:
preparing a framework composed of a material selected from the group consisting of magnesium metal and a magnesium-based metal alloy; and
forming a geometric design within the framework comprising a plurality of design features, comprising:
two or more fixation point openings each having a corresponding fixation connecting feature corresponding thereto; and
one or more non-fixation point openings each having a corresponding non-fixation connecting feature corresponding thereto,
wherein each of the corresponding fixation connecting feature and the corresponding non-fixation connecting feature surrounds and/or connects each of the two or more fixation point openings and the one or more non-fixation point openings, respectively, and
wherein each of the corresponding fixation connecting feature and the corresponding non-fixation connecting feature has a size that is structured to provide progressive and/or staged corrosion and/or absorption of the mesh.

17. A mesh, comprising:
a framework fabricated of a material selected from the group consisting of magnesium metal and a magnesium-based metal alloy; and
a geometric design formed within the framework comprising a plurality of design features, comprising:
two or more fixation point openings each having a corresponding fixation connecting feature corresponding thereto; and
one or more non-fixation point openings each having a corresponding non-fixation connecting feature corresponding thereto,
wherein each of the corresponding fixation connecting feature and the corresponding non-fixation connecting feature surrounds and/or connects each of the two or more fixation point openings and the one or more non-fixation point openings, respectively, and
wherein each of the corresponding fixation connecting feature and the corresponding non-fixation connecting feature has a size that is structured to provide progressive and/or staged corrosion and/or absorption of the mesh.

18. A method of making a mesh, comprising:
preparing a framework composed of a material selected from the group consisting of magnesium metal and a magnesium-based metal alloy; and
forming a geometric design within the framework comprising a plurality of design features, comprising:
two or more fixation point openings each having a corresponding fixation connecting feature corresponding thereto; and
one or more non-fixation point openings each having a corresponding non-fixation connecting feature corresponding thereto,
wherein each of the corresponding fixation connecting feature and the corresponding non-fixation connecting feature surrounds and/or connects each of the two or more fixation point openings and the one or more non-fixation point openings, respectively, and
wherein each of the corresponding fixation connecting feature and the corresponding non-fixation connecting feature has a size that is structured to provide progressive and/or staged corrosion and/or absorption of the mesh.
